(19) United States
(12) Patent Application Publication
(10) Pub. No.: US 2025/0342905 A1
(43) Pub. Date: Nov. 6, 2025

(54) SYSTEM AND METHOD FOR PROFILING BIOMOLECULES

(71) Applicant: Xin Gao, Hayward, CA (US)

(72) Inventor: Xin Gao, Hayward, CA (US)

(21) Appl. No.: 18/655,243

(22) Filed: May 4, 2024

(51) Int. Cl. G16B 15/30; G16C 20/30; G16C 20/50

(52) U.S. Cl. CPC G16B 15/30; G16C 20/30; G16C 20/50

(57) ABSTRACT

Presented is a process designed to provide a biomolecular profile for drug discovery endeavors. This process commences by receiving one or more compounds intended for addressing at least one disease. A biomolecular profile is initialized for a collection of biomolecules linked to the specified disease, weighing the relevance of each biomolecule to said disease. Further, the biomolecular profile undergoes updates contingent upon one or more quantifiable measures gauging the interaction between each compound and every biomolecule within the set.

### TECHNICAL FIELD

The present application relates to a system, apparatus, and method(s) of profiling biomolecules for drug discovery.

### BACKGROUND

Biomolecules such as proteins, DNA, and RNA work together in intricate networks to regulate biochemical processes, maintain cellular functions, and ensure the survival and reproduction of living organisms. Due to their fundamental importance as the building blocks of cellular processes, they play a pivotal role in drug discovery. Advances in our understanding of biomolecular structures, functions, and interactions continue to drive innovation in drug discovery and development, leading to the discovery of novel therapeutics for various diseases.

With the advent of machine learning and language models, significant innovations have emerged in drug discovery, particularly in modeling biomolecules within their respective biological pathways. However, challenges persist in identifying target compounds with both efficacy and low toxicity or off-target affinity. To tackle these challenges, our novel implementation of the biomolecular profile aims to provide a robust solution, improving various stages of the current drug discovery process.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features that facilitate the working of the invention and/or serve to achieve a substantially similar technical effect should be considered as falling into the scope of the invention disclosed herein.

The present disclosure introduces a biomolecular profile that provides valuable insights into how a compound behaves within biological systems and its potential impact on various biochemical pathways. This profile not only aids in identifying compounds with enhanced efficacy by comparing them to existing drugs on the market but also facilitates the improvement of existing drugs or the design of new ones in several ways. For instance, it assists in understanding potential drug toxicities, crucial for ensuring safety during clinical use. Specifically, the profile can evaluate drug candidates' toxicity or off-target affinity, encompassing classification for various toxicities such as genotoxicity, cardiotoxicity, hepatotoxicity, and other adverse effects. Furthermore, it helps elucidate a drug's interaction with other pathways related to pharmacokinetics, such as absorption, distribution, metabolism, and excretion properties. These properties influence the drug's bioavailability, tissue distribution, and elimination from the body. For example, the profile may utilize pharmacokinetic parameters as quantifiable measures to optimize dosing regimens and predict drug exposure levels in patients. The present invention encompasses various aspects that address the challenges.

In a first aspect, the present disclosure provides a method (or a computer-implemented method) for establishing a biomolecular profile for drug discovery, comprising: receiving one or more compounds for at least one disease; initiating a biomolecular profile for a set of biomolecules associated with said at least one disease based on a weighted relevance of each biomolecule of the set of biomolecules to said at least one disease; and updating the biomolecular profile based on one or more quantifiable measures of each compound of said one or more compounds interacting with or engaging each biomolecule of the set of biomolecules.

In a second aspect, the present disclosure provides a system comprising one or more computers and one or more storage devices storing instructions that when executed by the one or more computers cause one or more computers to perform steps of: receiving one or more compounds for at least one disease; initiating a biomolecular profile for a set of biomolecules associated with said at least one disease based on a weighted relevance of each biomolecule of the set of biomolecules to said at least one disease; and updating the biomolecular profile based on one or more quantifiable measures of each compound of said one or more compounds interacting with or engaging each biomolecule of the set of biomolecules.

In a third aspect, the present disclosure provides an apparatus comprising: at least one processor; and at least one memory including computer program code, wherein the at least one memory and the computer program code are configured, with the at least one processor, to cause the apparatus to perform method of the first aspect.

In a fourth aspect, the present disclosure provides a non-transitory computer-readable storage medium having stored thereon computer-readable code, which, when executed by computer apparatus, causes the computer apparatus to perform the method of the first aspect.

In a fourth aspect, the present disclosure provides a computer-readable medium comprising computer readable code or instructions stored thereon, which when executed on a processor, causes the processor to implement the method according to the first aspect.

The methods described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

### BRIEF DESCRIPTION OF THE DRAWINGS

Embodiments of the invention will be described, by way of example, with reference to the following drawings, in which:

FIG. 1 is a flow diagram of an example method for generating a biomolecular profile;

FIG. 2 is a block diagram of an example biomolecular profile and quantifiable measures;

FIG. 3 is a block diagram of an example compound modification based on optimizing for quantifiable measures of the biomolecular profile;

FIG. 4 is a block diagram of a system for using the biomolecular profile in drug discovery; and

FIG. 5 is a block diagram of an example computing environment.

### DETAILED DESCRIPTION

Embodiments of the present invention are described below by way of example only. These examples represent the suitable modes of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

A compound herein refers to a full or part of a substance or physical entity. The compound may be any organic or inorganic drug, which includes but are not limited to small molecules, biologics, and nucleic acid-based drugs. In drug discovery, a target compound represents an entity with potential therapeutic utility that undergoes extensive evaluation, optimization, and development to ultimately become the pharmaceutical drug or part of the pharmaceutical drug for treating one or more diseases that the compound targets.

Biomolecular profile comprises one or more quantifiable measures of a target compound's interactions at a molecular level with a set of biomolecules, which include but are not limited to, proteins, enzymes, receptors, and nucleic acids. For example, the biomolecular profile for one or more quantifiable measures may include but are not limited to measures such as the compound's binding affinity, specificity, mechanism of action, kinetics, metabolic stability, and indirectly quantifying the degree of toxicity.

This biomolecular profile may be represented as a statistical distribution, with a plurality of quantifiable measures of a target compound's interactions with biomolecules. As such, each quantifiable measure could be considered as a variable, and the distribution would represent the variability or distribution of values for each measure across different biomolecular interactions. Analysis of the distribution of biomolecular profile measures can help identify patterns, outliers, and correlations between different variables, leading to a better understanding of the compound's mode of action, efficacy, safety, and potential applications. Additionally, statistical methods can be applied to compare distributions between different compounds or conditions, aiding in the selection or optimization of candidate compounds for more efficient drug discovery.

Biomolecular profile may be updated, which refers to a biomolecular profile with at least one quantifiable measure calculated for most or all the compounds with the respect to each biomolecule in the profile. The updating process may be an iterative and ongoing process that involves integrating new data (either new compounds, addition of biomolecules, or based on other quantifiable measures), refining existing profiles, and communicating the updated profile model as described herein.

With respect to the biomolecular profile, binding affinity refers to the strength of interaction between the compound and the biomolecule(s), typically measured as binding affinity or dissociation constant (Kd). This indicates how tightly the compound binds to the biomolecule(s), which influences the compound's efficacy.

Specificity refers to the degree to which the compound selectively interacts with its intended target biomolecules as compared to other biomolecules (off-target) in the biological system, i.e. comparing the binding affinity of the compound to the target biomolecules versus its affinity to off-target biomolecules. This comparison can be expressed as selectivity ratios or similar metrics.

Metabolic stability refers to the rate at which a compound is metabolized in biological systems and can be quantified. For example, the extent of metabolic stability can be measured by determining the half-life of the compound in the presence of metabolic enzymes and calculating intrinsic clearance values.

Kinetics refers to the rate at which the compound binds to its target(s) biomolecules and the dynamics of the drug-target interaction, including association and dissociation rates. Kinetic parameters provide insights into the drug's onset of action, duration of effect, and potential for receptor desensitization or downregulation.

It is understood that the quantification of the above quantifiable measures or a combination thereof can be obtained using experimental data such as preclinical, in vitro data and animal data, or data from clinical trials, as well as a combination of experimental and experimental data generated from computational methods and algorithms.

Taking binding affinity for example, it may be obtained computationally using methods such as molecular docking, molecular dynamics simulation, quantum mechanics (QM), molecular mechanics (MM), a combination of QM/MM, free energy calculation, and other machine learning or statistical methods that help evaluate quantitative structure-activity relationship (QSAR).

Binding affinity can also be indirectly estimated or inferred from IC50 values, the concentration of a compound required to inhibit a biological process by 50%. For example, IC50 may also be obtained directly from binding assays and cell-based assays, and indirectly through methods such as surface plasmon resonance (SPR) and isothermal titration calorimetry (ITC).

Similar to binding affinity, other measures such as specificity, mechanism of action, kinetics, metabolic stability, and toxicity are readily determinable by quantitative methods empirically or from experimentation.

In the context of the biomolecular profile, weighted relevance (of each biomolecule of the set of biomolecules) involves assigning numerical weights to biomolecules based on their relevance or importance to the target disease, prioritizing certain biomolecules. This weighting scheme may be determined and calibrated via a combination of a knowledge base, which includes data from experiments, literature review, expert knowledge, and computational analyses. Further, the combination of information from the knowledge base for the target disease may be used to train an embedding for a large language model (LLM). The embedding captures and quantifies the relationship of each biomolecule to every other biomolecule as the weighted relevance. In effect, the relationship of a biomolecule in or part of a biological pathway of the target biomolecules would receive a higher weighted relevance than other biomolecules, while biomolecules in a different pathway would receive a relatively lower weighted relevance. The weighted relevance may be scaled and normalized in relation to different compounds. As such, the biomolecular profile is initiated for each compound based on the weighted relevance of each biomolecule to the disease. For example, an initiated biomolecular profile refers to a profile of compounds with their initial weighted relevance determined from or based on a knowledge base associated with the disease or obtained using experimental data or methods. As new data becomes available, such as quantifiable measures of compound interactions with biomolecules, the biomolecular profile may be updated to reflect changes in the relevance or importance of each biomolecule. This updating process ensures that the biomolecular profile remains current and reflects the most up-to-date understanding in terms of quantitative weights for association between the disease biology and potential therapeutic targets.

Similarity and dissimilarity measurements are generated with respect to the weighted relevance. Similarity measurement refers to a quantitative metric used to assess the similarity or resemblance between sets of data points, in this case biomolecular profiles. Similarity measurement can be based on Euclidean distance, cosine, Jaccard, Hamming distance, Pearson correlation, Levenshtein, or even some entropic methods. These same methods can also be used to generate dissimilarity measurement, which is a quantitative assessment of the dissimilarity sets of data points. Dissimilarity measurement quantifies how different or dissimilar two profiles are from each other based on the measurements. The degree to which compounds interact specifically with biomolecules associated with a disease may also be measured in terms of specificity or specificity measurement.

Overall, these measurements either rely on a weight relevance threshold or a weight relevance range in order to compare the biomolecular profiles of different compounds targeting the same disease, whether it is specificity, similarity, or dissimilarity. Different groups of biomolecules would have different measurements, which effectively provides a landscape of the biomolecules.

In one example, a subset of biomolecules falling within a dynamic range is deemed to have interactions with compounds that are neither too weak (below the threshold) nor too strong (above the threshold), indicating a level of specificity suitable for moving the compound forward. The measurement may consider a dynamic or pre-determined weighted relevance range instead a threshold value for each biomolecule. This range may be used to infer the acceptable level of interaction strength or relevance that a compound must achieve with a biomolecule to be considered with specificity.

The molecular profile may serve to train a profile model. The profile model may be any computational model or technique for analyzing and interpreting biomolecular interactions, guiding the selection and prioritization of compounds for drug discovery efforts. For example, the computation model or techniques may include but are not limited to one or more machine learning models, network models, statistical/probabilistic models, deep learning models, bayesian inference, language models, and graphical models.

By integrating different measures for the compound with respect to the biomolecules through experimental data, computational predictions, and other sources of information, the profile model helps researchers gain insights into the complex interplay between biomolecules and identify potential targets. Depending on the drug discovery goal, the profile model may be leveraged to generate the base biomolecular profile 208, either without considering the updated information or incorporating it to varying degrees, which allows easier backtesting. The base biomolecular profile 208, ineffective by updates, offers a reliable baseline comparison of any further profiles; it also offers more model stability, reducing bias and overfitting, as well as interpretability.

Profile model may be established on the basis of the conditional probability of each biomolecule interacting with or engaging every other biomolecule, which also refers to the likelihood or chance of a specific biomolecule interacting with another biomolecule given certain conditions. This probability quantifies the probability of an interaction occurring between any two biomolecules within a set of biomolecules, taking into account the influence of various factors such as molecular properties, environmental conditions, and biological context as captured by the quantifiable measures. The profile model may engage a synthesis model, utilizing information on biomolecular interactions and properties, for the generation of target compounds with specific properties or functions, starting from the profile model output or a deduction provided by the biomolecular profile.

Synthesis model refers to any computational framework designed to create synthesis pathways toward a target compound. It is customized according to the specific characteristics of the target compound, desired properties, and available resources. This model has the capability to predict and potentially improve current synthesis pathways to achieve optimal design. The model draws on concepts, including but not limited to retrosynthetic analysis and computer-assisted synthesis planning methods. Considering stereochemistry, the model may utilize strategies such as linear, convergent, divergent, and biomimetic synthesis routes, as well as click chemistry. Additionally, the model may incorporate techniques such as reaction prediction algorithms, molecular/quantum simulations, reaction network analysis, genetic algorithms in synthesis, predictions based on chemical properties, chirality prediction tools trained on a stereochemical database. By leveraging this array of approaches, chemists can effectively design and execute synthesis pathways tailored to their specific objectives and constraints. It is understood that various machine learning algorithms and artificial intelligence techniques may be applied and trained on large datasets of synthesis data to predict outcomes in reactions and molecular transformations, leveraging pattern recognition and statistics to make predictions of chemical or cellular synthesis pathways for producing the target compound in an optimal manner.

Molecular constituent refers to the components of a molecule. These constituents can include atoms, ions, functional groups, or other substructures that are bonded together to form larger molecules. In drug discovery or molecular design, molecular constituents refer to the specific chemical entities or motifs within a compound that contribute to its overall structure, properties, or biological activity.

While similar to molecular constituents, a functional group refers to specific groups of atoms within a molecule responsible for its characteristic chemical properties and reactivity. These groups impart distinct functionalities to the molecule, influencing its behavior in chemical reactions and interactions with other molecules. Functional groups often include elements such as carbon, hydrogen, oxygen, nitrogen, sulfur, and phosphorus, and they can range from simple groups like alkyl or hydroxyl to more complex ones like carbonyl, amino, or carboxyl. Examples of functional groups include the hydroxyl group in alcohols, the amino group in amines, and the carbonyl group in ketones and aldehydes.

In the context of this biomolecular profile, the score(s) herein described refers to a numerical value or comparable range assigned to quantify the strength, affinity, or efficacy of a compound's interaction with specific biomolecules or targets. This score is derived from various quantifiable measures such as binding affinity, activity, selectivity, or other relevant parameters assessed through experimental assays, computational predictions, or data analysis techniques. Essentially, the score represents the degree or extent of the compound's association or impact on the biomolecular targets included in the profile.

Quantifiable measure threshold refers to a predetermined value or range used as a criterion for filtering biomolecules based on their interactions with compounds. It indicates the minimum acceptable level of a quantifiable measure, such as binding affinity or activity, that a compound must achieve with each biomolecule to be considered relevant to the disease. Biomolecules that do not meet this threshold for interaction with at least one compound are filtered out, ensuring that only those biomolecules with meaningful interactions with compounds are included in the biomolecular profile.

Hierarchical data structure refers to a data structure that is adapted to organize the compounds based on their interactions with biomolecules and their potential relevance to targeting the disease. For example, the hierarchical data structure may involve categorizing compounds into different levels or tiers, with each level representing a different level of specificity or significance in terms of their interactions with biomolecular targets. These data structures may include but are not limited to trees, directed acyclic graphs, next lists, graph databases, and ontologies, or any data structures for the organization of multi-dimensional data as presented by a biomolecular profile comprising quantifiable measures M1 to Mn, shown in the figures.

Aggregation of scores refers to the process of combining or consolidating the scores assigned to compounds with respect to each biomolecule in the set. Various statistical methods may be used based on mean, weighted mean, medium, mode, trimmed mean. Other methods such as principal component analysis, k-means clustering hierarchical cluster, ensemble method, and deep learning models such as neural networks may be employed to obtain the aggregation of scores.

Consolidation of updated biomolecular profiles refers to the process of combining, organizing, or integrating information from the updated biomolecular profile based on groups of biomolecules. This can be achieved using statistical methods such as t-distributed stochastic neighbor embedding or multidimensional scaling, which can be used to visualize the biomolecular profile data in lower-dimensional space and identify clusters or groups of biomolecules based on their compound interaction profiles. Alternatively, other methods such as cluster, principal component analysis, graph-based algorithms may be used at various parts of the consolidation process.

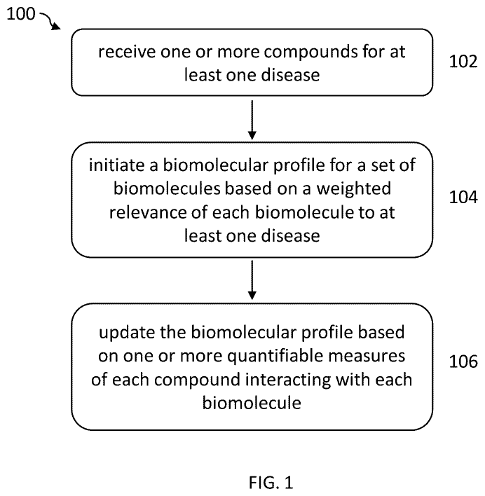

FIG. 1 shows method 100 for using a biomolecular profile and/or a profile model based on one or more biomolecular profiles for drug discovery. Various compounds associated with at least one disease are received or obtained to initiate the biomolecular profile based on a set of biomolecules pertinent to the disease. Each biomolecule's relevance to the disease is assessed and weighted accordingly, providing a structured foundation for further analysis with respect to one or more quantifiable measures. It is understood that the biomolecular profile may undergo refinement through continuous updates based on quantifiable measures of compound interactions with each biomolecule considered under the profile, i.e. the compound may directly interact, engage in certain biochemical or biophysical interactions with the biomolecular, or act a catalysis for the interaction with a difficult biomolecule. As such, the refinement process provides insights into the biomolecular landscape associated with the disease, facilitating the identification of potential drug candidates and elucidating their mechanisms of action. By integrating experimental data with biomolecular insights, this method empowers researchers to develop effective therapeutic interventions for complex diseases.

Moreover, the biomolecular profile could aid the researchers in understanding toxicity and evaluating drug interactions in various ways. By comparing a compound to known drugs via the biomolecule profile, the profile (or the profile model) can help classify the compound with respect to the biomolecules that cause toxic effects, i.e. identifying biomolecules associated with certain known toxicity such as genotoxicity, cardiotoxicity, and hepatotoxicity. This classification provides valuable insights into the potential safety profile of the compound, helping researchers prioritize compounds with lower toxicity risks for further development.

The biomolecular profile also allows researchers to analyze each compound's interaction with other pathways related to pharmacokinetics, including absorption, distribution, metabolism, and excretion properties. By elucidating these interactions, researchers can predict how the compound will behave in biological systems with respect to a comprehensive set of biomolecules in the human body, hence its bioavailability, tissue distribution, and elimination from the body. This information is crucial for optimizing dosing regimens, predicting drug exposure levels in patients, and minimizing the risk of adverse drug interactions. Following are examples of steps to obtain an updated biomolecular profile:

In step 102, receiving one or more compounds for at least one disease, where the compounds received may vary widely in their chemical structures, properties, and known or hypothesized biological activities. They could include small molecules, peptides, nucleic acids, or other types of molecules that have shown promise in preclinical or early-stage studies.

The selection of received compounds for inclusion may depend on various factors, i.e. starting from or based on a knowledge base associated with the disease, which includes information on the compounds' known or predicted mechanisms of action, their ability to target specific biomolecular pathways or targets implicated in the disease, their pharmacokinetic and pharmacodynamic properties, and any existing data on their safety and efficacy. Based on the knowledge base, a suitable set of biomolecules may be identified and selected for initiating the biomolecular profile. Alongside receiving the compounds, this set of biomolecules may also be received and embedded as part of the biomolecular profile.

Further optimizing the set of biomolecules ensures that only the most relevant biomolecules and interactions are retained for further analysis and interpretation. It helps prioritize biomolecular targets most likely to be involved in the disease mechanism or responsive to therapeutic intervention. The set of biomolecules is thereby filtered based on one or more quantifiable measures of at least one compound to each biomolecule in the set of biomolecules below a quantifiable measure threshold. The filtered set of the biomolecules is used to initiate the biomolecular profile or train a profile model as described herein.

In step 104, initiating a biomolecular profile for a set of biomolecules associated with said at least one disease based on a weighted relevance of each biomolecule of the set of biomolecules to said at least one disease, where each biomolecule is further evaluated for its relevance to the disease in a quantitative manner, considering more definitive factors such as biological function, involvement in disease pathways, and potential as a therapeutic target. These relevance assessments are weighted to reflect their relative importance, guiding the prioritization of biomolecules.

With this weighted relevance in mind, a biomolecular profile is initiated, capturing key information about the identity, relevance, and potential interactions of each biomolecule in the initial set of biomolecules or even after the initial set has been filtered as explained above. By focusing on a subset of the biomolecules, this biomolecular profile provides a foundational understanding of the biomolecular landscape associated with the disease, informing subsequent drug discovery efforts and therapeutic strategies to target the underlying molecular mechanisms.

In step 106, updating the biomolecular profile based on one or more quantifiable measures of each compound of said one or more compounds interacting with each biomolecule of the set of biomolecules, where further data on how each compound interacts with the biomolecules are incorporated. This step entails systematically assessing the interactions between each compound and each biomolecule, using measurable parameters such as binding affinity, specificity, inhibition potency, or other relevant metrics, also defined herein as quantifiable measures. These quantifiable measures provide insight into the strength and nature of the interactions, allowing for a more nuanced understanding of compound-biomolecule relationships. This may be an iterative process, where the biomolecular profile is continuously updated to remain dynamic and reflective of the latest experimental findings, guiding the selection and prioritization of potential drug candidates for further evaluation in the drug discovery pipeline.

The biomolecular profile may be used to train a profile model. The model is trained based on establishing the likelihood and/or conditional probability of interactions between each biomolecule in the set associated with the disease. This may be any computational model fit for the purpose. For example, it may be a probabilistic model that captures the inherent relationships between biomolecules, providing a framework for analyzing their interactions. The model may be updated based on the various iterations of the biomolecular profile, integrating new data and insights into compound-biomolecule interactions. Once trained, the profile model can be applied to analyze the biomolecular profile, extract patterns, and guide drug discovery efforts. Additionally, the model can be used to generate a base biomolecular profile either unweighted by the updated biomolecular profile or weighted by it, allowing for iterative refinement and comparison of the biomolecular profile over time. This comprehensive approach leverages probabilistic modeling techniques to gain insights into the complex biomolecular landscape associated with the disease.

Moreover, the profile model may be configured to identify one or more molecular constituents from the compounds meeting a threshold score based on a subset of the quantifiable measures and the biomolecules, where the threshold score may be a predefined value used to determine the significance or relevance of constituents. For example, the profile mode may assign scores to each compound based on a subset of quantifiable measures (such as binding affinity, kinetics, or degree of selectivity) and a subset of biomolecules (potentially representing specific targets or pathways), where the scores reflect the compound's potential relevance or effectiveness in interacting (or engage in any interaction/catalytic response) with the biomolecules. The profile model then applies a threshold score to filter out compounds that do not meet a certain level of significance or relevance. Compounds that surpass this threshold score are considered to have a sufficient level of interaction or activity with the biomolecules under consideration. The profile model may identify one or more molecular constituents from the compounds that meet the threshold score. These constituents are likely fragments, substructures, or functional groups within the compounds that contribute significantly to their interactions with the biomolecules, which forms the basis for lead compound synthesis. It is worth noting that the identification process is based on a subset of both quantifiable measures and biomolecules. This implies that the profile model may focus on specific aspects or characteristics of compounds and biomolecular interactions, rather than considering all available data. Overall, this approach allows the profile model to pinpoint molecular constituents within compounds that exhibit notable interactions with a subset of biomolecules.

It is understood that modifying the compound could be accomplished using the molecular constituents through an iterative process, which involves adding or removing molecular constituents from a compound in successive iterations. For example, the iterative addition or removal of molecular constituents (from a compound) may rely on the biomolecular profile and the synthesis model. The profile serves as a basis for identifying potential fragments or compounds. The synthesis model offers guidance on feasible modifications or additions to molecular structures based on chemical principles and synthetic feasibility. The iterative refinement process begins by proposing modifications to molecular constituents, considering insights from both the biomolecular profile and the synthesis model. After each iteration, the updated molecular constituents are evaluated within the context of the biomolecular profile, considering factors such as biological activity, interactions with biomolecules, and therapeutic relevance. Feedback from this evaluation informs adjustments to the proposed modifications, leading to further refinement. This iterative cycle continues until satisfactory candidates meeting the desired criteria for drug discovery, such as efficacy and safety, are identified.

Further steps may be taken to provide a comprehensive analysis of compound interactions with biomolecules using only the biomolecular profile, which allows precise identification of similarities, differences, and specificities in compound-target interactions across different compounds targeting the same disease, i.e. creating a new biomolecular profile for at least one additional compound potentially targeting the disease, where the profile is generated using the same approach as the initial biomolecular profile, utilizing the established biomolecular model. The second biomolecular profile is then compared to the updated biomolecular profile obtained from the initial compounds. This comparison involves analyzing the quantifiable measures of biomolecules in both profiles to identify similarities and differences in compound interactions.

Moreover, the similarity, dissimilarity, and specificity of compound interactions with biomolecules may be assessed in terms of the measurements. For example, the similarity measurement evaluates interactions of biomolecules with a weighted relevance above a threshold, identifying commonalities in interaction patterns across profiles. In another example, the dissimilarity measurement focuses on interactions of biomolecules with a weighted relevance below a threshold, highlighting differences in interaction patterns between profiles. In yet another example, the specificity measurement determines the degree of specificity of compound interactions with biomolecules, considering each biomolecule's weighted relevance within a certain range.

In practice, the similarity measurement would serve as a good indicator of the likeness or similarity of a compound to another compound while the dissimilarity measurement would determine difference in the same respect. The specificity measurement based on a range would be a more tailored approach for identifying and understanding a particular subset of molecular interactions. These measurements play key roles in various stages of drug discovery, from compound selection and screening to lead optimization, target identification, and especially toxicity prediction.

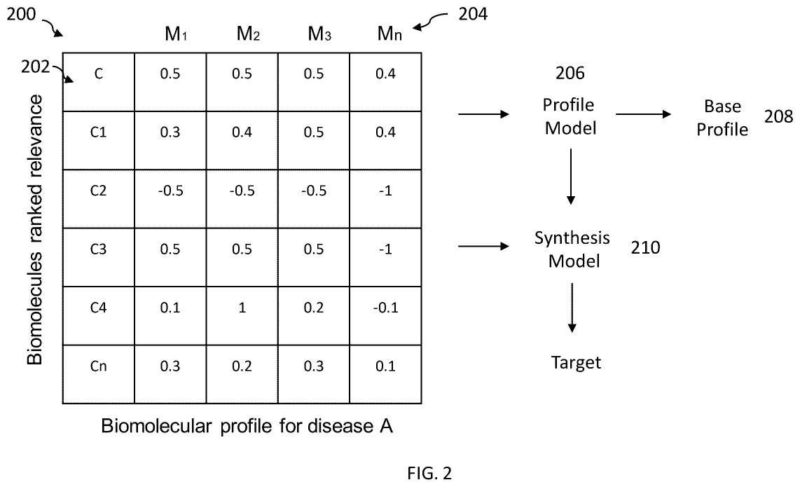

FIG. 2 is a block diagram showing biomolecular profile 200 for disease A with a quantifiable measure, i.e. binding affinity. The compounds 202 are shown as C to Cn. Biomolecules 204 that are related to the disease are shown as M1 to Mn, filtered and ranked based on weight relevance with M1>Mn. For example, the ranking may be based on predetermined scores signed to each biomolecule within them, representing their relevance to either the disease or to one another in the disease pathway. Various scoring methods can be used using experimental and expert knowledge, i.e. looking at the pathway of the disease, especially the up/down regulated protein starts, and applying statistical analysis to identify correlations, and machine learning models trained on labeled datasets. These scores serve to rank biomolecules based on their biological or biochemical relevance, guiding subsequent analyses or applications. In turn, these scores are then used to calculate the weighted relevance of each biomolecule, likely considering factors such as the magnitude of the score and the context of the biomolecule. Biomolecules that fall below a certain threshold of weighted relevance are removed from being added to the biomolecular profile. This selective filtering streamlines the dataset, retaining only the most relevant biomolecules for further analysis.

These scores may be aggregated as the number of compounds and biomolecules increases in order to be comparable. For example, an aggregate score may be obtained with respect to each biomolecule of the set of biomolecules. Individual scores for each compound can thereby be updated with respect to the aggregate score by normalizing the individual scores via the aggregate score to obtain a new score. Every new compound goes through this process to ensure that the scores are comparable.

In the figure, the biomolecular profile 200 is shown to be initiated and updated with respect to binding affinity scores as shown. The updated binding affinity is shown to be normalized and scored between −1 to 1. It is understood that the binding affinity for each compound may be an aggregation of a number of different binding affinity calculations acquired using various computation or experimental methods.

Compound C may be a known small molecular drug with market approval. M1 may be a protein that is known to be the target of compound C. M2 and M3 may be proteins in the biochemical pathway of M1, which may either up or down regulate M1. Mn may be a protein from a different pathway. As compared to the M1, Mn has a relatively high binding affinity score. This may be indicative of non-specific binding of the drug, which potentially could relate to the drug's side effects or reduction in efficacy.

Compound C1 has relatively similar scores as C for M1 to Mn, while C2 has different scores. A similarity measurement may be determined for C1 with respect to M1 to M3 and a dissimilarity measurement may be determined with respect to Mn. A range based on relevance may be selected for M2 and M3 only in terms of determining specificity measurement. These measurements would help classify the compounds based on similarity or difference to compound C, helping the identification of improved target candidates. For example, C3 appears to be a better target candidate with similarity for M1 to M3 and dissimilarity for Mn.

With the increase of compounds and biomolecules, statistical technical or computational models may be used, training on the data generated via the biomolecular profile to determine and compare compounds to known marketed drugs with respect to one or more quantifiable measures such as binding affinity.

Applying the computational model herein referred to as profile model 206 or using statistical methods to identify one or more targets would be feasible by comparing the relative similarity, dissimilarity, or specificity of the compound to the known or approved drug. The profile model 206 would be configured to generate a base biomolecular profile 208, which offers reliable baseline comparison of any further profiles, conferring obvious advantages such as model stability, reducing bias and overfitting, as well as interpretability. For example, a base biomolecular profile may be a foundational dataset, i.e. a compilation of measures for the initial set of biomolecules associated. The base biomolecular profile may provide a solid starting point for a research endeavor in understanding toxicity, and prioritizing compounds for further investigation to reduce off-target binding.

Moreover, the biomolecular profile 200 or profile model 206 may be paired with a synthesis model 208 trained to a certain class of synthesis. The synthesis model 208 comprises reaction knowledge at each step in optimally achieving a scalable quantity of the target compound while minimizing byproducts. It is understood that various types of synthesis models suitable for the present application are available and/or described herein.

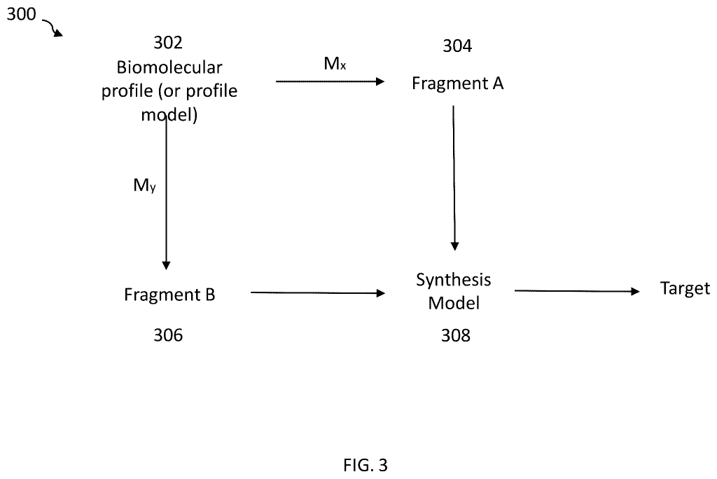

FIG. 3 is a block diagram of an example compound modification 300 based on optimizing for quantifiable measures of the biomolecular profile (or a profile model) 302. Starting with an updated biomolecular profile, based on the profile, various compounds may be identified and selected for assessment via an initiated or updated biomolecular profile. These compounds may be organized into a hierarchical data structure based on the updated biomolecular profile to reflect their relationships with one another. For example, compounds may be categorized according to relevant features or properties, involving defining hierarchical categories, assigning compounds to these categories based on their characteristics as determined by the biomolecular profile, and establishing parent-child relationships or nested levels within the hierarchy. Implementing a suitable data structure, such as a tree or nested list, facilitates efficient storage and retrieval of compound data while reflecting their relationships within the biomolecular profile. The hierarchical structure can be updated dynamically to accommodate changes or new insights in the biomolecular profile or compound dataset, ensuring alignment with the latest information for effective analysis and interpretation.

At least one compound that potentially targets the disease based on the hierarchical data structure may be identified, optionally based on the hierarchical data structure, which involves querying the structure to retrieve compounds associated with relevant disease-related categories or properties. Filters and criteria are then applied to narrow down the list based on the quantifiable measures, embedded in the updated biomolecular profile, capturing compound properties such as molecular similarity to known drugs and pharmacological properties of the compound as compared to other compounds. It is understood that compounds meeting these criteria are prioritized for further validation. These compounds may be fragments, and iteratively refined with the help of a synthesis model 308 as described herein and shown in the figure.

The figure shows fragments A 304 and B 306. Fragment A 304 is optimized quantitative measure Mx, while fragment B 306 is optimized for My. The synthesis model 308 aids in combining two fragments to form a target compound by providing guidance on the sequence of chemical reactions needed to achieve the desired transformation. For example, the model may take a systematic approach to pathway planning and optimization, a synthesis model may use fragment A 304 as the precursor fragments. The synthesis model formulates a synthetic pathway by analyzing the chemical transformations needed to convert fragment A into the target compound while incorporating fragment B 306. This involves selecting appropriate reactions and reagents based on principles of organic chemistry and synthetic feasibility. During this process, the model may use simulations to optimize the synthetic pathway, minimize the number of steps, maximize yield, or address other practical considerations around scaling. If the target compound is chiral or has specific stereochemical requirements, the synthesis model takes this into account during pathway planning. It ensures that the stereochemistry of the precursor fragments is preserved or controlled appropriately throughout the synthetic steps.

Overall, the synthesis model streamlines the process of starting from at least one fragment to form a target drug candidate, enabling robust and more iterative processes that may be considered alongside biomolecular profile 302. For example, the bottleneck or any efficiency in the synthesis identified by the model may be incorporated as a quantifiable measure in the biomolecular profile 302 or incorporated as part of the profile model described herein.

For example, the iterative process adapts to generate scores for each compound based on their interactions with biomolecules. By aggregating these scores across all compounds, a more comprehensive and comparable scoring system is established. This refinement ensures that compounds are evaluated more accurately and consistently based on their interactions with biomolecular targets. In subsequent iterations, a new biomolecular profile is generated, likely representing a different aspect or set of biomolecules. By comparing the scores from this new profile with the aggregated scores from previous iterations, similarities in scoring patterns are identified. Compounds that exhibit similar scores across multiple biomolecular profiles are considered to have desirable interactions across different biological contexts, indicating their potential as novel compounds for further investigation.

By continuously refining the scoring system and leveraging similarities in scores across iterations, this iterative process enhances compound identification by providing a more robust and nuanced understanding of their interactions with biomolecular targets. This approach increases the likelihood of identifying compounds with broad or specific bioactivity in relation to various quantitative measures as described herein.

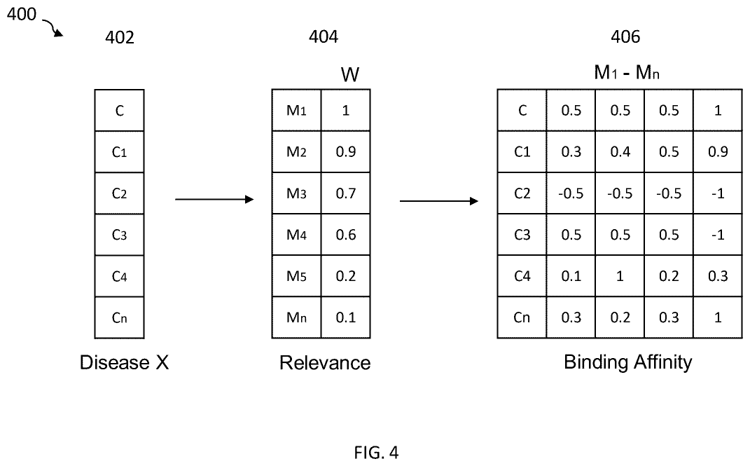

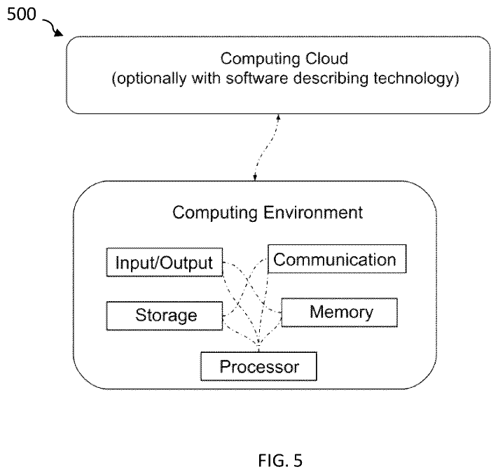

FIG. 4 is a block diagram of a system 400 for generating a biomolecular profile in drug discovery. The system may comprise one or more computers and one or more storage devices as shown in FIG. 5, storing instructions that, when executed by one or more computers cause one or more computers to receive 402 one or more compounds C to Cn for at least one disease X. Some of the compounds may be known drugs with market approval. These known drugs may retain data of the quantifiable measures, which may be weighted in relation to the other methods of obtaining the same quantifiable measures, whether empirically or based on experimental data.

In the next stage, the system would initiate a biomolecular profile 404 for a set of biomolecules M1 to Mn associated with at least one disease based on a weighted relevance W of each biomolecule of the set of biomolecules to said at least one disease. The weighted relevance may be normalized across all the biomolecules from the set, or an even larger collection of biomolecules based on the profile model of a disease, where a more comprehensive collection of biomolecules may be stored.

Finally, it will update the biomolecular profile 406 based on one or more quantifiable measures, i.e. binding affinity, of each compound of said one or more compounds interacting with each biomolecule of the set of biomolecules M1 to Mn. The updated biomolecular profile may be consolidated based on one or more groups of biomolecules based on the weighted relevance. For example, the biomolecules in the set (M1 to Mn) may be grouped into categories or clusters based on shared characteristics, functions, or biological pathways. This grouping facilitates the organization and interpretation of the biomolecular profile, allowing for a more structured analysis of compound interactions with related biomolecules. Consolidation may also take place based on the weighted relevance of the compound-biomolecule interactions. This weighting likely takes into account factors such as the importance of each biomolecule within its group, the magnitude of the quantifiable measures, or other considerations relevant to the specific application. In both cases, the consolidation process involves aggregating the updated information across all compounds and biomolecules within each group. This may involve averaging, summing, or otherwise combining the quantifiable measures to derive group-level metrics that represent the overall interaction profile for each compound within the group of biomolecules. Overall, the consolidated biomolecular profile provides a summarized view of compound interactions with different groups of biomolecules, highlighting key trends, similarities, or differences across biomolecular categories.

It is understood that the biomolecular profile or the profile model has various uses or applications throughout the drug discovery process. The biomolecular profile would play a valuable role in drug discovery by providing insights into not only affinity but also help elucidate toxicity mechanisms, facilitating the prediction and optimization of drug safety, and guiding the development of safer and more effective therapeutics. For example, by analyzing the interactions between compounds and biomolecules, the biomolecular profile can help predict potential off-target effects of drug candidates. This information is crucial for identifying compounds with undesirable side effects and optimizing drug design to minimize toxicity.

The biomolecular profile serves as a valuable screening tool for assessing the toxicity of compound libraries early in the drug discovery process. For instance, compounds from C1 to Cn may be selected based on their similarity to a subset of quantifiable measures related to toxicity and/or biomolecules identified through the biomolecular profile. Selected compounds are then modified to optimize these measures, aiming to reduce toxicity or off-target effects. Guided by a synthesis model, modifications such as adding or removing functional groups and/or molecular constituents are suggested to fine-tune the compound's properties. This iterative refinement process utilizes insights from the biomolecular profile and synthesis model to iteratively improve the compound's properties until desired optimization goals are achieved. The iterative refinement process ensures that the compound's design is optimized based on both its interactions with biomolecules (as indicated by the profile model) and its synthetic feasibility (as guided by the synthesis model), ultimately improving its potential effectiveness and reducing toxicity or off-target effects.

As shown in FIG. 5 the example computing environment 500 includes at least one processing unit and memory. In the figure, this most basic configuration is included within a dashed line. The processing unit executes computer-executable instructions and may be a real or a virtual processor. In a multi-processing system, multiple processing units execute computer-executable instructions to increase processing power and as such, multiple processors can run simultaneously. The memory may be volatile memory (e.g., registers, cache, RAM), non-volatile memory (e.g., ROM, FEPROM, flash memory, etc.), or some combination of the two. The memory stores software, images, and video that can, for example, implement the technologies described herein. A computing environment may have additional features. For example, one or more co-processing units or accelerators, including graphics processing units (GPUs), can be used to accelerate certain functions, including the implementation of CNNs and RNNs or any other ML techniques/models.

The computing environment is suitable for implementing various ML techniques or ML models as described herein, which includes any algorithms from labeled and/or unlabeled datasets such as data associated with the biomolecular. ML techniques include supervised methods like artificial neural networks (ANNs), decision trees, support vector machines (SVMs), and random forests, among others. Unsupervised ML techniques such as expectation-maximization (EM) algorithm and vector quantization are employed to infer hidden structures from unlabeled data. Additionally, semi-supervised ML methods, like active learning and graph-based methods, leverage both labeled and unlabeled datasets for training. Furthermore, the invention incorporates artificial neural network (ANN) ML techniques, including feedforward NNs, recurrent NNs (RNNs), and convolutional NNs (CNNs), as well as deep learning techniques such as deep belief networks and stacked autoencoders, to learn data representations from labeled and/or unlabeled datasets.ng environment.

Additionally, the computing environment is adapted to implement techniques associated with large language models and generative AI into drug discovery and pharmaceutical domain knowledge. By leveraging transformer-based models like GPT (Generative Pre-trained Transformer) and variational autoencoders (VAEs), the system enhances its understanding of pharmaceutical data and accelerates the discovery process. These techniques enable the model to analyze vast amounts of biomedical literature, extract relevant information, and generate hypotheses for drug candidates. Moreover, they facilitate the synthesis of new compounds by predicting molecular structures and properties based on learned patterns from existing datasets. This integration can improve the generation of weighted relevance for various biomolecules as well as derivative appreciate scores in relation to quantifiable measurements described herein.

The storage may be removable or non-removable and includes magnetic disks, magnetic tapes or cassettes, CD-ROMs, CD-RWs, DVDs, or any other medium which can be used to store information and that can be accessed within the computing environment. The storage stores instructions for the software, image data, and annotation data, which can be used to implement technologies described herein.

The computing environment may also include storage, one or more input device(s), one or more output device(s), and one or more communication connection(s). An interconnection mechanism (not shown) such as a bus, a controller, or a network, interconnects the components of the computing environment. Typically, operating system software (not shown) provides an operating environment for other software executing in the computing environment and coordinates the activities of the components of the computing environment.

The input device(s) may be a touch input device, such as a keyboard, keypad, mouse, touch screen display, pen, or trackball, a voice input device, a scanning device, or another device that provides input to the computing environment. For audio, the input device(s) may be a sound card or similar device that accepts audio input in analog or digital form, or a CD-ROM reader that provides audio samples to the computing environment. The output device(s) may be a display, printer, speaker, CD-writer, or another device that provides output from the computing environment.

The communication connection(s) enable communication over a communication medium (e.g., a connecting network) to another computing entity. The communication medium conveys information such as computer-executable instructions, compressed graphics information, video, or other data in a modulated data signal. The communication connection(s) are not limited to wired connections (e.g., megabit or gigabit Ethernet, Infiniband, Fibre Channel over electrical or fiber optic connections) but also include wireless technologies (e.g., RF connections via Bluetooth, WiFi (IEEE 802.11a/b/n), WiMax, cellular, satellite, laser, infrared) and other suitable communication connections for providing a network connection for the disclosed methods. In a virtual host environment, the communication(s) connections can be a virtualized network connection provided by the virtual host.

Some aspects of the disclosed methods can be performed using computer-executable instructions implementing all or a portion of the disclosed technology in a computing cloud. For example, disclosed compilers and/or processor servers are located in the computing environment, or the disclosed compilers can be executed on servers located in the computing cloud. In some examples, the disclosed compilers execute traditional central processing units (e.g., RISC or CISC processors).

Computer-readable media are any available media that can be accessed within a computing environment. By way of example, and not limitation, with the computing environment, computer-readable media include memory and/or storage. As should be readily understood, the term computer-readable storage media includes the media for data storage such as memory and storage and transmission media such as modulated data signals.

Aspects of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. aspects of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programable processor, a computer, or multiple processors or computers. The apparatus can include special purpose logic circuitry, e.g., an FPGA (field-programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other units suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, subprograms, or portions of code. A computer program can be deployed to be executed on one computer or on multiple computers that are located at one site or distributed across multiple sites and interconnected by a communication network.

As used in this specification, an “engine,” or “software engine,” refers to a software implemented input/output system that provides an output that is different from the input. An engine can be an encoded block of functionality, such as a library, a platform, a software development kit (“SDK”), or an object. Each engine can be implemented on any appropriate type of computing device, e.g., servers, mobile phones, tablet computers, notebook computers, music players, e-book readers, laptop or desktop computers, PDAs, smartphones, or other stationary or portable devices, that includes one or more processors and computer-readable media. Additionally, two or more of the engines may be implemented on the same computing device, or on different computing devices.

The processes and logic flow described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field-programmable gate array) or an ASIC (application-specific integrated circuit).

Computers suitable for the execution of a computer program include, by way of example, can be based on general or special purpose microprocessors or both, or any other kind of central processing unit. Generally, a central processing unit will receive instructions and data from a read-only memory or a random-access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; general-type disks; and CD-ROM and DVD-ROM disks as explained above. The processor and the memory can be supplemented by, or incorporated in, special purpose logic circuitry.

Aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of any invention or of what may be claimed, but rather as descriptions of features that may be specific to particular aspects of particular inventions. Certain features that are described in this specification in the context of separate aspects can also be implemented in combination in a single aspect. Conversely, various features that are described in the context of a single aspect can also be implemented in multiple aspects separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the aspects described above should not be understood as requiring such separation in all aspects, and it should be understood that the described program components and systems can generally be integrated together in a single software product or packaged into multiple software products.

Furthermore, the training or evaluation using neural networks as described herein are performed ideally using graphics processing units (GPUs) or field-programmable gate arrays (FPGAs). Of these GPUs are within the context of the computer or computers described in the above paragraphs. These GPUs can be hosted by a deep learning cloud platform such as Google Cloud Platform™. Examples of GPUs include but are not limited to Google's Tensor Processing Unit (TPU)™, NVIDIA DGX-1 ™ or Volta™ Microsoft′ Stratix V FPGA™, Graphcore's Intelligent Processor Unit (IPU)™, Qualcomm's Zeroth Platform™ with Snapdragon Processors™—Intel's Nirvana™, Movidius VPU™, Fujitsu DPI™ AP M's DynamicIQrm, IBM TrueNorth™. Preferably, GPUs will be used in combination of CPUs both for processing the networks as well as communicating with a number of peripheral devices via bus subsystem. These peripheral devices can include a storage subsystem including, for example, memory devices and a file storage subsystem, user interface input devices, user interface output devices, and a network interface subsystem.

Particular aspects of the subject matter have been described. Other aspects are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

Following aspects of the present invention are described with options that may be combined as appropriate with any one or more of the aspects or other options as described herein, as would be apparent to a skilled person. It is appreciated that any one or more options may be combined with any one or more preceding options or aspects.

One aspect is a method (or a computer-implemented method) for establishing a biomolecular profile for drug discovery, comprising: receiving one or more compounds for at least one disease; initiating a biomolecular profile for a set of biomolecules associated with said at least one disease based on a weighted relevance of each biomolecule of the set of biomolecules to said at least one disease; and updating the biomolecular profile based on one or more quantifiable measures of each compound of said one or more compounds interacting with or engaging each biomolecule of the set of biomolecules.

In another aspect is a system comprising one or more computers and one or more storage devices storing instructions that when executed by the one or more computers cause one or more computers to perform steps of: receiving one or more compounds for at least one disease; initiating a biomolecular profile for a set of biomolecules associated with said at least one disease based on a weighted relevance of each biomolecule of the set of biomolecules to said at least one disease; and updating the biomolecular profile based on one or more quantifiable measures of each compound of said one or more compounds interacting with or engaging each biomolecule of the set of biomolecules.

In another aspect is an apparatus comprising: at least one processor; and at least one memory including computer program code, wherein the at least one memory and the computer program code are configured, with the at least one processor, to cause the apparatus to perform any one or more aspect described herein.

In another aspect is a non-transitory computer-readable storage medium having stored thereon computer-readable code, which, when executed by computer apparatus, causes the computer apparatus to perform the method of the first aspect; or a computer-readable medium comprising computer readable code or instructions stored thereon, which when executed on a processor, causes the processor to implement the method according to any one or more aspect described herein.

As an option, further comprising: generating a second biomolecular profile of at least one second compound potentially targeting said at least one disease using the initiated biomolecular profile; and comparing the second biomolecular profile to the updated biomolecular profile for said at least one disease.

As another option, wherein said comparing the second biomolecular profile to the updated biomolecular profile for said at least one disease, further comprising: comparing said one or more quantifiable measures of one or more biomolecules of the set of biomolecules in the second biomolecular profile to one or more biomolecules of the updated biomolecular profile for said at least one disease. As another option, further comprising: generating a similarity measurement for at least part of said one or more biomolecules, wherein each biomolecule of said at least part of said one or more biomolecules with a weighted relevance above a weighted relevance threshold. As another option, further comprising: generating a dissimilarity measurement for at least part of said one or more biomolecules, wherein each biomolecule of said at least part of said one or more biomolecules with a weighted relevance below a weighted relevance threshold. As another option, further comprising: generating a specificity measurement for at least part of said one or more biomolecules, wherein each biomolecule of said at least part of said one or more biomolecules with a weighted relevance within a dynamic or pre-determined weight relevance range.

As another option, further comprising: initializing a profile model based on likelihood and conditional probability of each biomolecule interacting with every other biomolecule in the set of biomolecules; and updating the profile model based on the updated biomolecular profile. As another option, further comprising: applying the profile model; generating a base biomolecular profile unweighted by the updated biomolecular profile; or generating a first iteration of the base biomolecular profile weighted by the updated biomolecular profile.

As another option, further comprising: determining a score for each biomolecule of the biomolecular profile; determining the weighted relevance of each biomolecule based on the score; and removing a biomolecule that is below a threshold weighted relevance from the biomolecular profile.

As another option, further comprising: receiving a set of biomolecules related to the disease from a knowledge base; filtering the set of biomolecules based on said one or more quantifiable measures of at least one compound to each biomolecule in the set of biomolecules below a quantifiable measure threshold; and initiating the biomolecular profile with the filtered set of biomolecules.

As another option, further comprising: organizing said one or more compounds into a hierarchical data structure based on the updated biomolecular profile. As another option, further comprising: identifying at least one compound potentially targeting the disease based on the hierarchical data structure.

As another option, wherein said updating the biomolecular profile, further comprising: generating a score for each compound of said one or more compounds with respect to each biomolecule of the set of biomolecules; updating for each biomolecule of the set of biomolecules with the score weighted based on an aggregation of scores of said one or more compounds; generating a third biomolecular profile of at least one third compound potentially targeting said at least one disease using the initiated biomolecular profile; and identifying a third compound potentially targeting said at least one disease based on similarity in scores between updated biomolecular profile and the third biomolecular profile.

As another option, further comprising: identifying one or more molecular constituents from said one or more compounds meeting a threshold score based on a subset of said one or more quantifiable measures and a subset of the biomolecules.

As another option, further comprising: consolidating the updated biomolecular profile based on one or more groups of biomolecules based on the weighted relevance.

As another option, further comprising: selecting a compound based on the updated biomolecular profile with similarity in a set of quantifiable measures of said one or more quantifiable measures; and modifying the compound with respect to said one or more compounds based on optimizing for the set of quantifiable measures. As another option, wherein said modifying the compound comprising: adding or removing one or more molecular constituents to or from the compound or to arrive at the compound based on a synthesis model.

As another option, further comprising: identifying functional group for one or more functional group of each compound based on the updated biomolecular profile; classifying the set of compounds based on the identified functional group; and selecting a compound based on the classification optimizing for a set of quantifiable measures relevant to the identified functional group. As another option, further comprising: modifying the compound based on a synthesis model configured to Iteratively add or remove molecular constituents from the compound based on a profile model.

The described embodiments of the invention a system, process(es), method(s) and/or data structure and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

Those skilled in the art will realize that storage devices utilized to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item may also refer to one or more of those items. The term ‘comprising’ is used herein to mean including the method steps or elements identified, but that such steps or elements do not comprise an exclusive list and a method or apparatus may contain additional steps or elements.

The figures illustrate example methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, subroutines, programs, threads of execution, and/or the like. Still further, results of acts of the methods can be stored in a computer-readable medium, displayed on a display device, and/or the like.

The order of the steps of the methods described herein is an example, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A method for establishing a biomolecular profile for drug discovery, comprising:
receiving one or more compounds for at least one disease;
initiating a biomolecular profile for a set of biomolecules associated with said at least one disease based on a weighted relevance of each biomolecule of the set of biomolecules to said at least one disease; and
updating the biomolecular profile based on one or more quantifiable measures of each compound of said one or more compounds interacting with each biomolecule of the set of biomolecules.

2. The method of claim 1, further comprising:
generating a second biomolecular profile of at least one second compound potentially targeting said at least one disease using the initiated biomolecular profile; and
comparing the second biomolecular profile to the updated biomolecular profile for said at least one disease.

3. The method of claim 2, wherein said comparing the second biomolecular profile to the updated biomolecular profile for said at least one disease, further comprising:
comparing said one or more quantifiable measures of one or more biomolecules of the set of biomolecules in the second biomolecular profile to one or more biomolecules of the updated biomolecular profile for said at least one disease.

4. The method of claim 3, further comprising:
generating a similarity measurement for at least part of said one or more biomolecules, wherein each biomolecule of said at least part of said one or more biomolecules with a weighted relevance above a weighted relevance threshold.

5. The method of claim 3, further comprising:
generating a dissimilarity measurement for at least part of said one or more biomolecules, wherein each biomolecule of said at least part of said one or more biomolecules with a weighted relevance below a weighted relevance threshold.

6. The method of claim 3, further comprising:
generating a specificity measurement for at least part of said one or more biomolecules, wherein each biomolecule of said at least part of said one or more biomolecules with a weighted relevance within a dynamic or pre-determined weight relevance range.

7. The method of claim 1, further comprising:
Initializing a profile model based on likelihood and conditional probability of each biomolecule interacting with every other biomolecule in the set of biomolecules; and
updating the profile model based on the updated biomolecular profile.

8. The method of claim 7, further comprising:
applying the profile model; generating a base biomolecular profile unweighted by the updated biomolecular profile; or
generating a first iteration of the base biomolecular profile weighted by the updated biomolecular profile.

9. The method of claim 1, further comprising:
determining a score for each biomolecule of the biomolecular profile;
determining the weighted relevance of each biomolecule based on the score; and
removing a biomolecule that is below a threshold weighted relevance from the biomolecular profile.

10. The method of claim 1, further comprising:
receiving a set of biomolecules related to the disease from a knowledge base;
filtering the set of biomolecules based on said one or more quantifiable measures of at least one compound to each biomolecule in the set of biomolecules below a quantifiable measure threshold; and
initiating the biomolecular profile with the filtered set of biomolecules.

11. The method of claim 1, further comprising:
organizing said one or more compounds into a hierarchical data structure based on the updated biomolecular profile.

12. The method of claim 11, further comprising:
identifying at least one compound potentially targeting the disease based on the hierarchical data structure.

13. The method of claim 1, wherein said updating the biomolecular profile, further comprising:
generating a score for each compound of said one or more compounds with respect to each biomolecule of the set of biomolecules;
updating for each biomolecule of the set of biomolecules with the score weighted based on an aggregation of scores of said one or more compounds;
generating a third biomolecular profile of at least one third compound potentially targeting said at least one disease using the initiated biomolecular profile; and
identifying a third compound potentially targeting said at least one disease based on similarity in scores between updated biomolecular profile and the third biomolecular profile.

14. The method of claim 1, further comprising:
identifying one or more molecular constituents from said one or more compounds meeting a threshold score based on a subset of said one or more quantifiable measures and a subset of the biomolecules.

15. The method of claim 1, further comprising:
consolidating the updated biomolecular profile based on one or more groups of biomolecules based on the weighted relevance.

16. The method of claim 1, further comprising:
selecting a compound based on the updated biomolecular profile with similarity in a set of quantifiable measures of said one or more quantifiable measures; and
modifying the compound with respect to said one or more compounds based on optimizing for the set of quantifiable measures.

17. The method of claim 16, wherein said modifying the compound comprising:
adding or removing one or more molecular constituents to or from the compound or to arrive at the compound based on a synthesis model.

18. The method of claim 1, further comprising:
identifying functional group for one or more functional group of each compound based on the updated biomolecular profile;
classifying the set of compounds based on the identified functional group; and
selecting a compound based on the classification optimizing for a set of quantifiable measures relevant to the identified functional group.

19. The method of claim 18, further comprising:
modifying the compound based on a synthesis model configured to iteratively add or remove molecular constituents from the compound based on a profile model.

20. A system comprising one or more computers and one or more storage devices storing instructions that when executed by the one or more computers cause one or more computers to perform steps of:
receiving one or more compounds for at least one disease;
initiating a biomolecular profile for a set of biomolecules associated with said at least one disease based on a weighted relevance of each biomolecule of the set of biomolecules to said at least one disease; and
updating the biomolecular profile based on one or more quantifiable measures of each compound of said one or more compounds interacting with each biomolecule of the set of biomolecules.
